(19) United States
(12) Patent Application Publication
(10) Pub. No.: US 2024/0366514 A1
(43) Pub. Date: Nov. 7, 2024

(54) PHARMACEUTICAL COMPOSITIONS OF OZANIMOD

(71) Applicant: ZYDUS LIFESCIENCES LIMITED, Ahmedabad (IN)

(72) Inventors: Kannan Essakimuthu MUTHAIYYAN, Ahmedabad (IN); Chetan Rajashekhara MURTHY, Ahmedabad (IN); Ritesh Harbans KAPOOR, Ahmedabad (IN); Rajesh Bharat MAHESHWARI, Ahmedabad (IN); Jay Shantilal KOTHARI, Pennington, NJ (US)

(73) Assignee: ZYDUS LIFESCIENCES LIMITED, Ahmedabad (IN)

(21) Appl. No.: 18/578,843

(22) PCT Filed: Jul. 13, 2022
(86) PCT No.: PCT/IB2022/056442

§ 371 Date: Jan. 12, 2024

(30) Foreign Application Priority Data
Jul. 14, 2021 (IN) 202121031569

(51) Int. Cl. A61K 9/48; A61K 9/20; A61K 9/28; A61K 31/4245

(52) U.S. Cl. CPC A61K 9/4866; A61K 9/2018; A61K 9/2027; A61K 9/2054; A61K 9/282; A61K 9/2846; A61K 9/4858; A61K 9/4891; A61K 31/4245

(57) ABSTRACT

The present invention relates to pharmaceutical compositions of ozanimod or a pharmaceutically acceptable salt thereof. In particular, the invention relates to pharmaceutical compositions of ozanimod or a pharmaceutically acceptable salt thereof, one or more pharmaceutically acceptable excipients, and optionally one or more controlled release substances. The invention also relates to processes for the preparation of such compositions and use thereof for treatment of inflammatory bowel disease.

### FIELD OF THE INVENTION

The present invention relates to pharmaceutical compositions of ozanimod or a pharmaceutically acceptable salt thereof. In particular, the invention relates to pharmaceutical compositions of ozanimod or a pharmaceutically acceptable salt thereof and one or more controlled release substances. The invention also relates to processes for the preparation of such compositions and use thereof for treatment of inflammatory bowel disease.

### BACKGROUND OF THE INVENTION

Ozanimod or 5-(3-{(1S)-1-[(2-hydroxyethyl)amino]-2,3-dihydro-1H-inden-4-yl}-1,2,4-oxadiazol-5-yl)-2-[(propan-2-yl)oxy]benzonitrile, is represented by the chemical formula;

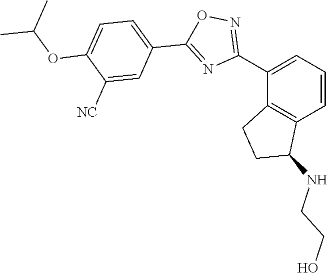 [SMILES: CC(C)Oc1ccc(-c2nc(-c3cccc4c3CC[C@@H]4NCCO)no2)cc1C#N]

U.S. Pat. Nos. 8,481,573 and 8,796,318 disclose ozanimod and its related compounds, along with their pharmaceutically acceptable salts as well as treatment of a diseases mediated by S1P1 activation.

Ozanimod is approved in the US under the brand Zeposia® as an immediate release capsule form with doses of 0.23 mg, 0.46 mg, and 0.92 mg once daily for the treatment of multiple sclerosis. Although ozanimod is a selective S1P modulator, it is associated with certain dose related adverse events including cardiac toxicity. To control the dose related toxicity, the dose of Zeposia® needs to be escalated from 0.23 mg to recommended maintenance dose of 0.92 mg once daily starting on Day 8.

Inflammatory bowel disease is a chronic immune-mediated inflammatory disorder that often treated with immune-suppressants and immune system modulators, with the intent to dampen aberrant immune responses. Inflammatory bowel disease, including ulcerative colitis and Crohn's disease are chronic gastrointestinal inflammatory disorders characterized by infiltration of lymphocytes, macrophages, and other immune cells. Inflammatory bowel disease is debilitating autoimmune disease that causes chronic diarrhea. One of the disease's defining characteristics is an abnormal accumulation of lymphocytes or T-cells in the lining of the gut. This activation of immune cells causes inflammation resulting in chronic, painful bowel movements. There are certain approved treatment options for the treatment of irritable bowel diseases which includes budesonide, mesalamine, glucocorticoids, thiopurines, and some biological agents such as TNF-α agonist (infliximab, adalimumab, and golimumab) and the anti-integrin (vedolizumab). However, these treatment options are found inadequate in the treatment of ulcerative colitis and Crohn's disease.

The sphingosine-1-phosphate (S1P) receptors are responsible for regulating multiple immunologic and cardiovascular effects. Cell surface associated S1P1-receptor plays a crucial role in the trafficking of lymphocytes from lymphoid organs. S1P1-receptor agonists induce internalization and degradation of the S1P1 receptor, rendering B and T lymphocytes incapable of migrating from secondary lymphoid organs, which leads to a reversible reduction in circulating lymphocytes in the blood.

Several researchers have attempted to evaluate safety and/or efficacy of various drugs and therapeutic approaches in order to find better treatment options for inflammatory bowel disease.

Montrose et al., (J Lipid Res. 2013 March; 54(3):843-851) studied the role of S1P1 on blood vessels in the colon. It has been observed that S1P1 is overexpressed in the colonic mucosa of ulcerative colitis patients. This increase in S1P1 levels reflected increased vascular density in the inflamed mucosa. In addition to playing a critical role in controlling immune cell egress, S1P receptors also regulate vascular function. They are strongly expressed on endothelial cells, with S1P1 being the most abundant of the receptors. Montrose et al., discusses that ulcerative colitis is a disease of the colorectum whereby patients manifest cyclical bouts of inflammation, which can result in severe morbidity. The damage that occurs in the colonic mucosa of ulcerative colitis patients is associated with an intense lymphocytic influx. The colonic vasculature also plays an important role in the pathogenesis. During inflammation, vascular density increases, and blood vessels become more permeable coincident with immune cell extravasation and infiltration of the colonic mucosa. Abnormal vasculature in ulcerative colitis is believed to contribute to the chronic inflammatory state which damages the colon.

Argollo et al., (Expert Opin. Biol. Ther. 20 (4)) discusses that S1P-receptor modulators act as functional antagonists at lymphocytic S1P1-receptors, inhibit S1P1 receptor-dependent lymphocyte egress from the secondary lymphoid organs to the periphery and decrease numbers of circulating lymphocytes including auto-reactive T cells, therefore, finally resulting in immunomodulatory effects. Argollo et al., found that the novel class of drug, S1P modulators from the new generation of oral small molecules represents a milestone on the evolution of therapeutic class of medicines followed by two decades of monoclonal antibodies (anti-TNF, vedolizumab, and ustekinumab). S1P modulators benefit from convenient oral administration, short half-life, no immunogenicity, with great therapeutic potential in ulcerative colitis.

Thus, there is an existing and continual unmet need of a pharmaceutical composition which releases ozanimod in a predetermined manner such that the dose related adverse effect can be minimized along with desired therapeutic outcome in the treatment of inflammatory bowel disease.

### SUMMARY OF THE INVENTION

In one general aspect, the present invention provides a pharmaceutical composition comprising ozanimod and one or more pharmaceutically acceptable excipients.

In another general aspect, the present invention provides a pharmaceutical composition comprising ozanimod, one or more controlled release substances, and one or more pharmaceutically acceptable excipients.

In another general aspect, the present invention provides a pharmaceutical composition comprising: i) a core composition comprising ozanimod, one or more pharmaceutically acceptable excipients, and optionally one or more controlled release substances; and ii) a coating comprising one or more controlled release substances.

In one another aspect, the present invention provides a method of treating ulcerative colitis comprising administering to a subject in need thereof an effective amount of a pharmaceutical composition comprising ozanimod, one or more controlled release substances, and one or more pharmaceutically acceptable excipients selected from diluent, binder, and lubricants; wherein the composition exhibits a dissolution profile such that not less than 25% of ozanimod is released in 1 hour and not less than 75% ozanimod is released in 6 hours, when tested in a U.S.P. Type II apparatus (paddles) at 37° C. and 50 rpm, in pH 6.8 phosphate buffer.

In another general aspect, the present invention provides a pharmaceutical composition in the form of a matrix comprising ozanimod and one or more controlled release substances, the matrix composition is optionally coated with one or more coating materials.

In one another aspect, the present invention provides a delayed-release pharmaceutical composition comprising ozanimod, one or more controlled release substances, and one or more pharmaceutically acceptable excipients selected from diluent, binder, and lubricants; wherein the pharmaceutical composition releases not less than 40% of ozanimod after 15 minutes and not less than 65% of ozanimod after 60 minutes when subjected to a test medium comprising 500 mL of 6.8 phosphate buffer at 37° C. in a standard USP basket apparatus at 100 rpm.

In one another aspect, the present invention provides a pharmaceutical composition comprising a multi-particulate composition comprising ozanimod, one or more controlled release substances, and one or more pharmaceutically acceptable excipients, wherein the composition initiates to release ozanimod in a dissolution media having a pH of about 5 to about 7.5.

In one general aspect, the present invention provides a pharmaceutical composition comprising: i) a core composition comprising ozanimod; and ii) a coating composition comprising pH dependent polymer, wherein the composition comprises a sub-coating layer between the core and the coating.

In one general aspect, the present invention provides a pharmaceutical composition comprising two types of particles comprising ozanimod, wherein the first and the second type of particles release ozanimod as discreet pulses separated by a period of time.

In a further general aspect, the present invention provides a pharmaceutical composition comprising ozanimod, wherein ozanimod is released from the composition as a first and a second dose, wherein the first and the second doses are released from the composition as discreet pulses of ozanimod separated by a period of time, and wherein the second dose contains equal or more amount of ozanimod than the first dose.

In yet another general aspect, the present invention provides a pharmaceutical composition comprising ozanimod, wherein ozanimod is released from the composition as a first and a second dose, and wherein the first and the second doses are released from the composition as discreet pulses of ozanimod separated by a period of time, wherein the second dose contains equal or more amount of ozanimod than the first dose and begins release of the dose between 1 and 6 hours after the first dose begins to be released.

In still another general aspect, the present invention provides a method of treating ulcerative colitis in a patient in need of treatment thereof, administering a therapeutically effective amount of the pharmaceutical composition comprising at least two types of particles containing ozanimod, wherein the first type of particle releases ozanimod at a pH of about 5.0 to about 5.5 and the second type particle releases ozanimod at a pH of about 6.2 to about 7.5; wherein the first type of particles and second type of particles are present in a ratio of 1:9 to 9:1 in the pharmaceutical composition.

In another general aspect, the present invention provides a process of preparing a pharmaceutical composition comprising ozanimod and one or more pharmaceutically acceptable excipients.

In yet another aspect, the present invention provides use of a pharmaceutical composition comprising ozanimod and one or more pharmaceutically acceptable excipients in the treatment of ulcerative colitis.

The details of one or more embodiments of the invention are set forth in the description below. Other features, objects and advantages of the invention will be apparent from the description.

### BRIEF DESCRIPTION OF THE DRAWINGS

FIG. 1 shows effect of ozanimod formulations, i.e., Pharmacodynamic parameters of Ozanimod after oral administration of Immediate-Release (IR) formulation and Delayed-Release (DR) formulations of Ozanimod in 2,4,6-Trinitrobenzenesulfonic acid (TNBS) induced irritable bowel disease (IBD) in Wistar rat male model, A) Colon weight to length ratio, B) Macroscopic Colon Disease Score, and C) Circulating Lymphocyte count.

### DETAILED DESCRIPTION OF THE INVENTION

The inventors have developed pharmaceutical compositions comprising ozanimod, one or more controlled release substances, and one or more pharmaceutically acceptable excipients which can deliver a therapeutically effective amount of ozanimod in a controlled manner. Such compositions result in an effective treatment of irritable bowel disease and offer better treatment to patients.

The term “ozanimod” should be understood, unless otherwise indicated herein, to include pharmaceutically acceptable salts, the corresponding racemates, diastereoisomers, enantiomers, tautomers, as well as the corresponding crystal modifications, e.g., solvates, hydrates and polymorphs.

The articles “a”, “an” and “the” are used to refer to one or to more than one (i.e., to at least one) of the grammatical object of the article.

Inflammatory bowel disease (IBD) is an umbrella term used to describe disorders that involve chronic inflammation of digestive tract. Types of IBD include Ulcerative colitis and Crohn's disease. Both ulcerative colitis and Crohn's disease usually involve severe diarrhea, abdominal pain, fatigue and weight loss. IBD can be debilitating and sometimes leads to life-threatening complications. Immune system's overactive response is believed as one of the important factors in development of Ulcerative colitis. The various clinical studies established that S1P1 is overexpressed in the colonic mucosa of ulcerative colitis patients. This increase in S1P1 levels reflected increased vascular density in the inflamed mucosa which damages the colon. Ozanimod is a novel, selective, oral S1P1-receptor and S1P5-receptor modulator. It binds to receptors on the cells' surface, diminishing the immune attack. The inventors of the present invention developed pharmaceutical compositions which release ozanimod in a controlled manner for exerting anti-inflammatory activity resulting in rapid and effective care of the disease symptoms. The inventors of the present invention developed modified release pharmaceutical compositions for delivering therapeutically effective amount of ozanimod which help in efficient management of ulcerative colitis symptoms.

The pharmaceutical compositions of the present invention are solid oral dosage forms. The solid dosage form includes, for example, tablets, caplets, capsules, pills, granules, or powder filled in sachets, and the like. The compositions may be further coated with film forming agents suitable for immediate and/or modified release profiles.

The term “modified-release” may be considered to include controlled-release, sustained-release, delayed-release, and timed-release profile. A “delayed-release” composition may be designed to delay the release of the drug for a specified period.

In one of the embodiments, the present invention provides a pharmaceutical composition comprising a pharmaceutically effective amount of ozanimod, one or more controlled release substances, and one or more pharmaceutically acceptable excipients.

The term “therapeutically effective amount” refers to the amount of active compound or pharmaceutical agent that elicits the biological or medicinal response in a tissue, system, animal, individual or human that is being sought by a researcher, veterinarian, medical doctor or other clinician or caregiver or by an individual, which includes at least one of the following:
1. Preventing the disease, for example, preventing a disease, condition or disorder in an individual that may be predisposed to the disease, condition or disorder but does not yet experience or display the pathology or symptomatology of the disease;
2. Inhibiting the disease, for example, inhibiting a disease, condition or disorder in an individual that is experiencing or displaying the pathology or symptomatology of the disease, condition or disorder (i.e., arresting further development of the pathology and/or symptomatology); and
3. Ameliorating the disease, for example, ameliorating a disease, condition or disorder in an individual that is experiencing or displaying the pathology or symptomatology of the disease, condition, or disorder (i.e., reversing the pathology and/or symptomatology).

According to general embodiment of the present invention, the pharmaceutical composition may be in the form of monolithic dosage from or multi-particulate dosage form. Such dosage form may include tablets, mini-tablets, granules, capsules, and sachets. The pharmaceutical composition may comprise about 0.05% w/w to about 25% w/w, preferably 0.1% w/w to 15% w/w, more preferably about 0.5% w/w to 5% w/w of ozanimod.

According to one another embodiment of the present invention, it provides a delayed-release pharmaceutical composition comprising ozanimod, one or more controlled release substances, and one or more pharmaceutically acceptable excipients.

In yet another embodiment of the present invention, it provides a delayed-release pharmaceutical composition comprising ozanimod, one or more controlled release substances, and one or more pharmaceutically acceptable excipients; wherein the composition releases ozanimod at a pH of about 5 to about 7.5.

In yet another embodiment, the present invention provides a delayed-release pharmaceutical composition comprising ozanimod, one or more controlled release substances, and one or more pharmaceutically acceptable excipients selected from diluent, binder, and lubricants; wherein the pharmaceutical composition releases less than 40% of ozanimod after 15 minutes, not less than 50% of ozanimod after 30 minutes, not less than 60% of ozanimod after 45 minutes, and not less than 65% of ozanimod after 60 minutes when subjected to a test medium comprising 500 mL of 6.8 phosphate buffer at 37° C. in a standard USP basket apparatus at 100 rpm.

In one of the embodiments of the present invention, it has now been found that multi-particulate formulations are particularly more suitable, since the particles are having large surface area to release active compound in a more controlled manner and are thus particularly suitable for the treatment of inflammatory conditions. In order to achieve desired therapeutic action, the active compound must get release from the composition at or near the site of inflammation within a relatively short time and continue to be release for quite some longer time, preferably 4 to 8 hours, more preferable up to 12 hours in order that its action does not wear off too rapidly.

Hence, in one of the aspects, the present invention provides a multi-particulate pharmaceutical composition comprising ozanimod, one or more controlled release substances, and one or more pharmaceutically acceptable excipients.

In one of the embodiments, the pharmaceutical composition comprises multi-particulate compositions comprising a) a core comprising ozanimod and one or more pharmaceutically acceptable excipients, and b. a coating over the core, wherein the coating comprises one or more controlled release substances which initiate release of ozanimod at a pH of about 5.

In one of the general embodiments, the present invention provides a delayed-release pharmaceutical composition comprising i) a core composition comprising ozanimod or a pharmaceutically acceptable salt thereof and one or more pharmaceutically acceptable excipients, and ii) a coating comprising one or more controlled release substances.

In yet another embodiment, the present invention provides a delayed-release pharmaceutical composition comprising i) a core composition comprising ozanimod or a pharmaceutically acceptable salt thereof and one or more pharmaceutically acceptable excipients, and ii) a coating comprising one or more controlled release substance, wherein the pharmaceutical composition releases not less than 40% of ozanimod after 15 minutes, not less than 50% of ozanimod after 30 minutes, not less than 60% of ozanimod after 45 minutes, and not less than 65% of ozanimod after 60 minutes when subjected to a test medium comprising 500 mL of 6.8 phosphate buffer at 37° C. in a standard USP basket apparatus at 100 rpm.

In yet another embodiment, the present invention provides a multi-particulate pharmaceutical composition comprising ozanimod, one or more pharmaceutically acceptable excipients and optionally, one or more controlled release substances; wherein the composition releases ozanimod as a first and a second dose, wherein the first and second doses are released from the composition as discreet pulses separated by a period of time, wherein the second dose contains equal or more amount of ozanimod than the first dose.

In one of the general embodiments, the pharmaceutical composition is in the form of capsules containing multi-particulate compositions comprising ozanimod, one or more pharmaceutically acceptable excipients, and optionally, one or more controlled release substances. The multi-particulate composition may comprise single type, two types, or multiple types of particles having distinct drug release profile. The multi-particulate compositions may be in a coated or uncoated form having an immediate-release and/or a modified-release profile.

In another embodiment, the pharmaceutical composition comprises two or more type of particles. Preferably, the pharmaceutical composition comprises at least two types of particles having distinct release profiles. The pharmaceutical composition comprises two types of particles, the first type of particles having a modified-release profile and the second type of particles having an immediate-release profile.

In yet another embodiment, the pharmaceutical composition comprises two types of modified-release particles; wherein the first type of particles release ozanimod at a pH of about 5.0 to about 5.5 and the second type of particles release ozanimod at a pH of about 6.2 to about 7.5, wherein the first type of particles and the second type of particles are present in a ratio of from about 1:9 to 9:1 in the pharmaceutical composition.

In one of the general embodiments, the present invention provides a pharmaceutical composition comprising two types of particles, the first type of particles having a modified-release profile and the second type of particles having an immediate-release profile; wherein the modified-release particles release ozanimod at a pH about 5 to about 7.5, and wherein the first type of particles and the second type of particles are present in a ratio of from about 1:9 to 9:1.

In one another embodiment, the present invention provides a capsule composition comprising two types of particles; the first type of particles having a modified-release profile and the second type of particles having an immediate-release profile; wherein the modified release particles release ozanimod at a pH about 5 to about 7.5; and wherein the first type of particles and the second type of particles are present in a ratio of from about 1:9 to 9:1.

In the pharmaceutical compositions, the particles may be termed interchangeably as granules, pellets, mini-tablets, and like. The particles may be present in the form of coated or uncoated form. Preferably, the pharmaceutical composition is a tablet or a capsule. The particles may comprise ozanimod dispersed in a controlled release substance matrix which may be further coated with a functional or non-functional coating material.

In one of the embodiments, the composition comprises ozanimod in an amount ranging from about 0.05 mg to 2 mg. Preferably, the composition comprises ozanimod in an amount of about 0.050 mg, 0.075 mg, 0.1 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.875 mg, 0.900 mg, 0.925 mg, 0.950 mg, 0.975 mg, 1.000 mg, 1.125 mg, 1.250 mg, 1.375 mg, 1.500 mg, 1.625 mg, 1.750 mg, 1.875 mg, and 2.000 mg.

In one of the embodiments, the capsules may be in the form of coated capsules. The capsules are coated with at least one controlled release substances for releasing ozanimod at a pH of about 6.2 to 7.5. The capsules are coated with a controlled release substance which dissolves at a pH of about 5.0, for example at a pH of about 5.5, for example at a pH of about 6, for example at a pH of about 6.8.

In one of the general embodiments, the controlled release substance may be a pH dependent polymer, such as a cellulose derivative, for example hydroxypropyl methylcellulose phthalate, hydroxypropyl methylcellulose acetate succinate, cellulose acetate phthalate, and the like, acrylate polymers, such as methacrylic acid and methyl methacrylate based polymers, and the like; pH independent polymers, such as polyvinyl alcohol, polyvinyl acetate, copolymer of polyvinyl acetate and polyvinylpyrrolidone, polymethacrylic acid derivatives, cellulose derivatives, such as ethyl cellulose, hydroxypropyl methylcellulose, hydroxypropyl cellulose, and the like; Polyethylene Oxide; chitosan and its derivative; gums such as guar gum, polysaccharides, such as pectin and amylose; lipids, waxes, cyclodextrins, polyols, and likes. The controlled release substance may be used as a matrix former along with the active substance or may be used suitably for coating the core comprising active substance. The controlled release substance used in the present composition may be in a range of from about 1% w/w to about 75% w/w of the total composition.

The particles may be prepared by direct compression, dry granulation, or wet granulation process. The particles may also be prepared by extrusion and/or spheronization process, or multi-layering of composition components on a nonpareil seeds. The particles of the present invention are having size, D90 less than 1000 microns, preferably D₉₀ less than 750 microns, more preferable D90 less than 500 microns.

In one of the general embodiments, the present invention provides a multi-particulate pharmaceutical composition prepared by a process comprising:
(a) a core comprising ozanimod and one or more pharmaceutically acceptable excipients,
(b) a first coating layer over the core (a) comprising one or more controlled release substances,
(c) optionally, an intermediate coating layer on the layered core (b),
(d) a second coating layer comprising ozanimod over core (b) or over intermediate layer coated core (c), if present, and
(e) a third coating layer on core (d) comprising at least one controlled release substance.

In another embodiment, the method of preparing a modified release ozanimod particles comprise: a) providing nonpareil seeds of substantially uniform size; b) providing ozanimod dispersion, and optionally binder dispersion; c) layering said core with the ozanimod dispersion simultaneously with or after optional layering of said core with the binder dispersion to provide ozanimod core. The drug coated particles optionally coated with sub-coting layer comprising amino methacrylate copolymer, such as Eudragit® E polymers, followed by coating with one or more controlled release substances.

According to one embodiment, a pharmaceutical composition is prepared by a fluidized bed processor, process comprising:
(a) Ozanimod, one or more diluent, one or more pharmaceutically acceptable excipients, and optionally, a controlled release substance blended uniformly in a suitable blender,
(b) binder is dispersed in a granulation solvent to obtain a uniform dispersion,
(c) uniform blend obtained in step (a) is granulated by spraying dispersion of step (b),
(d) granules are dried at a temperature of about 40° C.,
(e) dried granules are sized by passing through a suitable sieve,
(f) the sized granules are optionally coated with a coating composition comprising one or more controlled release substances,
(g) granules are lubricated with a suitable lubricant and are either filled into capsules or compressed into tablets using suitable tablet tooling, and
(h) capsule or tablet compositions are optionally coated with a coating composition comprising one or more controlled release substances.

In another embodiment, a delayed-release pharmaceutical composition is prepared by a process comprising:
a) providing nonpareil seeds of substantially uniform size;
b) preparing aqueous and/or nonaqueous dispersion comprising ozanimod and optionally binder;
c) layering said nonpareil seeds with the dispersion of step (b) to form ozanimod core;
d) drying the core until desired LoD is achieved;
e) coating the dried pellet of step (d) with film coating material to obtain intermediate film coated pellets:
f) drying the pellets of step (e) until desired LoD is achieved;
g) preparing aqueous and/or nonaqueous dispersion comprising controlled release substances
h) coating the dried ozanimod pellets of step (f) with dispersion of step (g);
i) drying the coated pellets of step (h) until desired LoD is achieved;
j) optionally, curing the pellets of step (i) at 45+5° C.;
k) optionally, lubricating the pellets of step (j) with a lubricant; and
l) filling the pellets of step (j) or (k) it into capsules;
m) optionally, coating the filled capsules of step (1) with a coating composition comprising controlled release substance.

Pulsed release systems, the other broad category of modified release dosage forms, are also well known in the art. Pulsed release systems generally involve a first drug release and a second drug release separated by a predetermined period of time or site of release. Pulsed release systems also may include a combination of immediate-release and extended-release systems. Multiple formulation configurations are suitable for pulsed release dosage forms.

Multiparticulate systems have also been proposed for purposes of providing a pulsed release of drug. Such systems typically use distinct populations of drug containing particles to achieve a pulsed release. The populations employ different coating polymers, such as those mentioned above, to release the drug at different points in time or location. For example, polymers having different dissolution pHs are commonly used for this purpose. Hence, one type of particles can be coated with a polymer that begins dissolving at a pH of about 5.5 to 6 and another type of particles can be coated with a polymer that begins dissolving at a pH of 6.5 to 7.5 for achieving a pulsed release. In this manner, the first type of particles would release the drug in the upper small intestine while the second type of particles would release the drug further downstream and therefore at a later time.

Thus, in one of another embodiments, the present invention provides a pharmaceutical composition comprising two types of particles comprising ozanimod, one or more pharmaceutically acceptable excipients, and optionally, one or more controlled-release substances; wherein the first and the second type of particles release ozanimod as discreet pulses separated by a period of time.

In one of the general embodiments, the invention provides a pharmaceutical composition comprising ozanimod, one or more pharmaceutically acceptable excipients and optionally, one or more controlled-release substances; wherein ozanimod is released from the composition as a first and a second dose; wherein the first and second doses are released from the composition as discreet pulses of ozanimod separated by a period of time, wherein the second dose contains equal or more amount of ozanimod than the first dose.

In a further embodiment, the pharmaceutical composition may begin release of the second dose between 1 and 6 hours after the first dose begins to be released.

In a matrix system, the drug is homogenously dispersed in one or more controlled release substances, and optionally one or more pharmaceutically acceptable excipients. This admixture is typically compressed under pressure to produce a tablet. Drug is released from the tablet by diffusion and erosion. Matrix systems typically employ a controlled release substance, such as a water-soluble hydrophilic polymer, or a water insoluble hydrophobic polymer (including waxes). Examples of suitable water-soluble polymers include polyvinylpyrrolidine, hydroxypropylcellulose, hydroxypropylmethyl cellulose, methyl cellulose, vinyl acetate copolymers, polysaccharides (such as alignate, xanthum gum, pectin, chitosan, amylose), polyethylene oxide, methacrylic acid copolymers, maleic anhydride/methyl vinyl ether copolymers and derivatives, and mixtures thereof. Examples of suitable water insoluble polymers include acrylates, cellulose derivatives such ethylcellulose or cellulose acetate, polyethylene glycols, methacrylates, acrylic acid copolymers and high molecular weight polyvinylalcohols. Examples of suitable waxes include fatty acids and glycerides. The controlled release substance used in the present composition may be in a range of from about 1% w/w to 50% w/w of the composition.

In another embodiment, the pharmaceutical compositions of the present invention may be in the form of a monolithic matrix type tablet dosage form. A matrix type tablet comprises ozanimod, one or more controlled release substances, and one or more pharmaceutically acceptable excipients, wherein the tablet may be further coated with a coating composition comprising a controlled release substance.

The matrix tablets are prepared by direct compression, wet granulation, or a dry granulation process. In a preferred embodiment, wet granulation is a preferred method for preparing the tablet.

Diluents, or fillers, can be added to, for example, to increase the mass of an individual dose to a size suitable for tablet compression. Suitable diluents include, for example, powdered sugar, calcium phosphate, calcium sulfate, microcrystalline cellulose, lactose, mannitol, kaolin, sodium chloride, dry starch, and sorbitol. The amount diluent used in the pharmaceutical composition may be in a range of about 10% w/w to 90% w/w of the composition.

Lubricants are incorporated into a formulation for a variety of reasons. They reduce friction between the granulation and die wall during compression and ejection. This prevents, for example, granules blend from sticking to the tablet punches, and facilitates its ejection from the tablet punches. Examples of suitable lubricants include talc, stearic acid, vegetable oil, calcium stearate, zinc stearate, and magnesium stearate.

Glidants can also be incorporated into a formulation, typically for purposes of improving the flow characteristics of the granulation. Examples of suitable glidants include talc, silicon dioxide, and cornstarch.

Binders also may be incorporated into the formulation. Examples of suitable binders include povidone, polyvinylpyrrolidone, cellulose gums such as carboxymethylcellulose, methyl cellulose, hydroxypropylmethylcellulose, hydroxycellulose, gelatin, and starch.

The nonpareil seeds may be selected from sugar sphere, cellulose spheres, or pH modifying agent such as tartaric acid, succinic acid, citric acid, and the like.

Suitable granulating solvents may include purified water, methanol, ethanol, isopropyl alcohol, dichloro methane, acetone, or combinations thereof.

In another embodiment, the pharmaceutical composition of the invention may be further coated with a functional or non-function film coating material.

The functional coating comprises pH dependent or pH independent controlled release substance for modifying the drug release from a pharmaceutically acceptable composition. In certain embodiments the coating composition comprises either any one or a mixture of pH dependent and pH independent controlled release substances for obtaining the desired release profile.

The controlled release substance may be pH dependent polymers, such as cellulose derivatives, for example hydroxypropyl methylcellulose phthalate, hydroxypropyl methylcellulose acetate succinate, and the like, acrylate polymers such as methacrylic acid and methyl methacrylate based polymers, and the like; pH independent polymers, such as polyvinyl alcohol, polyvinyl acetate, copolymer of polyvinyl acetate and polyvinylpyrrolidone, polymethacrylic acid derivatives, polyethylene oxide, cellulose derivatives, such as ethyl cellulose, hydroxypropyl methylcellulose, hydroxypropyl cellulose, and the like; chitosan and its derivative; gums, such as guar gum, polysaccharides such as pectin and amylose; lipids, waxes, cyclodextrins, and the likes.

The non-functional coating, film coating or intermediate coating may comprise material like hydroxypropyl cellulose, hydroxyethyl cellulose, hydroxypropyl methylcellulose, polyvinyl pyrrolidone, copolymer of vinyl pyrrolidone and vinyl acetate; plasticizers like polyethylene glycol, triacetin, dibutyl sebecate and diethyl tartrate; opacifying agents like titanium dioxide and talc; and coloring agents. Examples of such non-functional coats are commercially available Opadry® compositions. Such coating may be also of polyol such as mannitol, sugar, or polyethylene glycol.

In one of the embodiments, the composition may comprise one or more muco-adhesive polymers, so as to retain composition in the gastrointestinal tract for a longer duration of time. The non-limiting examples of bio-adhesive polymers are carboxymethyl cellulose, polyacrylic acid polymers, amino methacrylate copolymer, polycarbophil, cellulosics, chitosan, diethylaminodextran, diethylaminoethyldextran, polygalactosamine, polylysine, polyomithine, prolamine, polyimine, hyaluronic acid, sodium alginate, sodium carboxymethylcellulose (sodium CMC), alginate and like.

Suitable examples of plasticizers include, but not limited to glycerin fatty acid esters; triethyl citrate; propylene glycol; polyethylene glycol and the like.

Other excipients that may be incorporated into the formulation include preservatives, antioxidants, or any other pharmaceutically acceptable excipient.

In one of the embodiments, the invention provides a pharmaceutical composition comprising ozanimod, one or more controlled release substances, and one or more pharmaceutically acceptable excipients, wherein the composition exhibits a dissolution profile such that not less than 25% of ozanimod is released in 1 hour and not less than 75% ozanimod is released in 6 hours, when tested in a U.S.P. Type II apparatus (paddles) at 37° C. and 50 rpm, in pH 6.8 phosphate buffer.

In one of the general embodiments, the present invention provides a method of administering a pharmaceutical composition to a subject in need thereof an effective amount of a pharmaceutical composition comprising ozanimod for the treatment of irritable bowel disease.

In one another embodiment, there is provided a method of treating irritable bowel disease comprising administering to a subject in need thereof an effective amount of a pharmaceutical composition comprising ozanimod, at least one controlled release substance, and at least one pharmaceutically acceptable excipient selected from diluent, binder, and lubricants, wherein the composition exhibits a dissolution profile such that not less than 25% of ozanimod is released in 1 hour and not less than 75% ozanimod is released in 6 hours, when tested in a U.S.P. Type II apparatus (paddles) at 37° C. and 50 rpm, in pH 6.8 phosphate buffer.

In another general embodiment, there is provided a stable pharmaceutical composition comprising ozanimod and at least one pharmaceutically acceptable excipient, wherein the composition retains at least 95% of the initial amount of ozanimod after storage for 3 months at 40° C. and 75% relative humidity.

In an embodiment, the present invention includes use of packaging materials such as containers and lids of high-density polyethylene (HYPE), low-density polyethylene (LDPE) and or polypropylene or polyethylene and/or glass, and blisters or strips composed of aluminum or high-density polypropylene, polyvinyl chloride, polyvinylidene dichloride, or aluminium/aluminium foil blisters or polyvinyl chloride/polyethylene/polyvinylidene dichloride (PVC/PE/PVDC) film packaging.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### EXAMPLES

The present invention is explained in detail in the following by referring to the examples, which are not to be construed as limitative.

### Capsules Composition

### Example 1: Preparation of Immediate-Release (IR) Granules

Hydroxypropylmethyl cellulose (HPMC 3cps) is dispersed in purified water and mixed well. Ozanimod, sodium starch glycolate, and talc are dispersed in the above HPMC dispersion and mixed well. The predetermined amount of ozanimod containing solution is applied on to sugar spheres (nonpareil seeds) using a fluidized bed processor. After the completion of coating, the granules are dried under vacuum at 40° C. until desired loss on drying (LOD) is achieved.

TABLE 1
Composition of immediate-release granules

| Ingredients | IR Granules (Ex. 1a) | | IR Granules (Ex. 1b) | |
|---|---|---|---|---|
| Sugar Spheres NF | 140 | mg | 70 | mg |
| Ozanimod | 1 | mg | 0.5 | mg |
| HPMC 3 cps | 3 | mg | 1.5 | mg |
| Sodium Starch Glycolate | 2 | mg | 1 | mg |
| Talc | 2.5 | mg | 1.25 | mg |
| Purified water | q.s. | | q.s. | |
| Total | 148.5 | mg | 74.25 | mg |

### Example 2: Delayed-Release (DR) Granules Prepared by Coating Immediate-Release Granules Core with Delayed-Release Polymer

Triethyl citrate is dispersed in methacrylic acid co-polymer dispersion and mixed well. Talc is dispersed in purified water and mixed well. Mix both of the above dispersions together to get uniform dispersion and then purified water is added to get approximately 10-15% w/w dispersion. The immediate-release granule according to Ex. 1a and Ex. 1b are coated with the above prepared delayed-release coating dispersion using a fluidized bed processor. The delayed-release coated granules are dried at 40° C. for predetermined time.

TABLE 2
Composition of DR granules prepared by coating IR granules core

| | | |
|---|---|---|
| | DR Granules | DR Granules |
| Ingredients | (Ex. 2a) | (Ex. 2b) |

| | | | |
|---|---|---|---|
| IR Granules (Ex. 1a) | 148.5 | mg | — |

| | | | |
|---|---|---|---|
| IR Granules (Ex. 1b) | — | 74.25 | mg |

| | | | | |
|---|---|---|---|---|
| Methacrylic Acid Co-polymer | 26.12 | mg | 13.06 | mg |
| Triethyl Citrate | 2.1 | mg | 1.05 | mg |
| Talc | 11.70 | mg | 5.85 | mg |

| | | |
|---|---|---|
| Purified water | q.s. | q.s. |

| | | | | |
|---|---|---|---|---|
| Total | 188.42 | mg | 94.21 | mg |

### Example 3: Preparation of Sustained Release (SR) Granules

Ethyl cellulose aqueous dispersion is dispersed in acetyltributyl citrate and mixed well. Ozanimod and polysorbate 80 are dispersed in purified water. Both of the above dispersions are mixed together to get uniform dispersion. The predetermined amount of ozanimod containing solution is sprayed on to sugar spheres (nonpareil seeds) using a fluidized bed processor. After the completion of coating, the granules are dried under vacuum at 40° C. until desired loss on drying (LOD) achieved.

TABLE 3
Composition of sustained-release (SR) granules

| | | |
|---|---|---|
| | SR Granules | SR Granules |
| Ingredients | (Ex. 3a) | (Ex. 3b) |

| | | | | |
|---|---|---|---|---|
| Sugar Spheres NF | 140 | mg | 70 | mg |
| Ozanimod | 1 | mg | 0.5 | mg |
| Ethylcellulose aq. dispersion | 7 | mg | 3.5 | mg |
| Acetyltributyl citrate | 0.5 | mg | 0.25 | mg |

| | | | |
|---|---|---|---|
| Polysorbate 80 | 0.06 | mg | 0.03 |

| | | |
|---|---|---|
| Purified water | q.s. | q.s. |

| | | | | |
|---|---|---|---|---|
| Total | 148.56 | mg | 74.28 | mg |

### Example 4: Delayed-Release (DR) Granules Prepared by Coating Sustained-Release Granules Core with Delayed-Release Polymer

Triethyl citrate is dispersed in methacrylic acid co-polymer dispersion and mixed well. Talc is dispersed in purified water and mixed well. Mix both of the above dispersions together to get uniform dispersion and then purified water is added to get approximately 10-15% w/w dispersion. The sustained-release granule according to Ex. 2a and Ex. 2b are coated with the above prepared delayed-release coating dispersion using a fluidized bed processor. The delayed-release coated granules are dried at 40° C. for predetermined time.

TABLE 4
Composition of DR granules prepared by coating SR granules core

| | | |
|---|---|---|
| | DR Granules | DR Granules |
| Ingredients | (Ex. 4a) | (Ex. 4b) |

| | | | |
|---|---|---|---|
| SR Granules (Ex. 3a) | 148.56 | mg | — |

| | | | |
|---|---|---|---|
| SR Granules (Ex. 3b) | — | 75.28 | mg |

| | | | | |
|---|---|---|---|---|
| Methacrylic Acid Co-polymer | 26.12 | mg | 13.06 | mg |
| Triethyl Citrate | 2.1 | mg | 1.05 | mg |
| Talc | 11.64 | mg | 5.82 | mg |

| | | |
|---|---|---|
| Purified water | q.s. | q.s. |

| | | | | |
|---|---|---|---|---|
| Total | 188.42 | mg | 94.21 | mg |

### Example 5: Preparation of Capsules Composition Comprising Delayed-Release IR Core Granules

The delayed-release granules prepared according to Ex. 2a are lubricated using about 1.58 mg of talc, and the lubricated delayed-release granules are filled in capsules.

### Example 6: Preparation of Capsules Composition Comprising Delayed-Release SR Core Granules

The delayed-release granules prepared according to Ex. 4a are lubricated using about 1.58 mg of talc, and the lubricated delayed-release granules are filled in capsules.

### Example 7: Capsules Composition Comprising DR Granules Containing IR Core and DR Granules Containing SR Core

The DR granules prepared according to Ex. 2b are lubricated using 0.79 mg of talc. Separately, the DR granules prepared according to Ex. 4b are lubricated using 0.79 mg of talc. Both type of above lubricated granules are mixed together and are filled in capsules in weight ratios as given in table 7 below;

TABLE 7
Capsules composition comprising

| | Composition | | Composition | | Composition | | Composition | |
|---|---|---|---|---|---|---|---|---|
| | 1 | | 2 | | 3 | | 4 | |
| Lubricated DR | 75 | mg | 50 | mg | 25 | mg | 100 | mg |
| Granules (Ex. | | | | | | | | |
| 2b) | | | | | | | | |
| Lubricated DR | 125 | mg | 150 | mg | 175 | mg | 100 | mg |
| Granules (Ex. | | | | | | | | |
| 4b) | | | | | | | | |
| Capsule Fill | 200 | mg | 200 | mg | 200 | mg | 200 | mg |
| weight | | | | | | | | |
| | Composition | | Composition | | Composition | | Composition | |
| | 5 | | 6 | | 7 | | 8 | |
| Lubricated DR | 125 | mg | 150 | mg | 175 | mg | 20 | mg |
| Granules (Ex. | | | | | | | | |
| 2b) | | | | | | | | |
| Lubricated DR | 75 | mg | 50 | mg | 25 | mg | 180 | mg |
| Granules (Ex. | | | | | | | | |
| 4b) | | | | | | | | |
| Capsule Fill | 200 | mg | 200 | mg | 200 | mg | 200 | mg |
| weight | | | | | | | | |

### Example 8: Capsules Composition Comprising Multilayer Delayed-Release Ozanimod Granules

TABLE 8

| Ingredients | Amount | |
|---|---|---|
| Sugar Spheres NF | 150 | mg |
| Drug Layering | | |
| Ozanimod | 1.0 | mg |
| HPMC 3 cps | 3.0 | mg |
| Tartaric Acid | 0.03 | mg |
| Purified Water | q.s. | |
| Weight of Drug Layered granule | 154.03 | mg |
| Sustained Release Layer | | |
| Ethyl Cellulose | 7.0 | mg |
| Acetyltributyl Citrate | 0.5 | mg |
| Purified Water | q.s. | |
| Weight of SR granule | 161.53 | |
| Mucoadhesive Layer | | |
| Amino Methacrylate Copolymer | 10.0 | mg |
| Talc | 0.1 | mg |
| Purified Water | q.s. | |
| Weight of mucoadhesive layered granule | 171.63 | |
| Delayed-Release Layer | | |
| Methacrylic acid ethyl acrylate | 26.5 | mg |
| Triethyl Citrate | 2.0 | mg |
| Talc | 8.0 | mg |
| Purified Water | q.s. | |
| Weight of DR Granules | 208.13 | mg |
| Lubrication | | |
| Talc | 1.5 | mg |
| Total Weight | 209.63 | mg |

A) Preparation of Drug Layered Granules: Hydroxypropylmethyl cellulose (HPMC 3cps) is dispersed in purified water and mixed well. Ozanimod and tartaric acid are dispersed in the above HPMC dispersion and mixed well. The predetermined amount of ozanimod containing solution is sprayed on to sugar spheres (nonpareil seeds) using fluidized bed processor. The coated granules are dried under vacuum at 40° C. until desired loss on drying (LOD) is achieved.
B) Preparation of Sustained-Release (SR) Granules: Acetyltributyl citrate is dispersed in ethylcellulose aqueous dispersion and mixed well. The drug layered granules are coated with the above dispersion using a fluidized bed processor. The coated granules are dried under vacuum at 40° C. till obtaining the desired.
C) Preparation of Mucoadhesive Substance Layered Granules: Amino methacrylate copolymer and talc are mixed in purified water and mixed well. The SR granules are coated with above dispersion using a fluidized bed processor. The obtained granules are dried under vacuum at 40° C. until desired loss on drying (LOD) is achieved.
D) Preparation of Delayed-Release Granules: Methacrylic acid ethyl acrylate, triethyl citrate, and talc are dispersed in purified water and mixed well. The mucoadhesive substance layered granules are coated with the above dispersion using a fluidized bed processor. The obtained granules are dried under vacuum at 40° C. until desired loss on drying (LOD) is achieved.
E) Lubrication of Delayed-Release Granules: The DR granules are lubricated using talc is ready for filling in capsules.

### Example 9: Delayed-Release Tablets Composition

Ozanimod, lactose monohydrate, hydroxypropyl methylcellulose 100 cps, and microcrystalline cellulose are blended together in rapid mixer granulator (RMG). Separately, povidone K 30 is dissolved in purified water to prepare a binder solution. The binder solution is added in to above ozanimod-excipients blend in RMG to form granules. The wet granules are dried and passed through co-mill fitted suitable screen to obtained the desired size granules. The dried granules are mixed with microcrystalline cellulose, colloidal silicone dioxide and magnesium stearate. The above blend is compressed in to tablets on the tablet press.

The delayed-release coating dispersion is prepared by dispersing together methacrylic acid co-polymer, triethyl citrate, talc, and Polysorbate 80 in purified water and mixed well. The compressed tablets are coated with delayed-release coating and dried at 40° C. for suitable time.

TABLE 9

| Ingredients | Quantity/Tablet | |
|---|---|---|
| Intra-granular Part | | |
| Ozanimod | 1 | mg |
| Lactose Monohydrate | 30 | mg |
| Hydroxypropyl Methylcellulose 100 cps | 50 | mg |
| Microcrystalline Cellulose | 20 | mg |
| Povidone K 30 | 2.5 | mg |
| Purified water | q.s. | |
| Extra-granular Part | | |
| Microcrystalline Cellulose | 20 | mg |
| Colloidal Silicone Dioxide | 2 | mg |
| Magnesium Stearate | 2 | mg |
| Delayed- Release Coating | | |
| Methacrylic Acid Copolymer | 9 | mg |
| Triethyl Citrate | 1 | mg |
| Polysorbate 80 | 1 | mg |
| Talc | 4 | mg |
| Purified Water | q.s. | |
| Total Weight of tablet | 122.5 | mg |

### Example 10: Delayed-Release Capsules Compositions

Hydroxypropylmethyl cellulose was dispersed in purified water and mixed well. Ozanimod was dispersed in methanol and mixed well to get a solution and was added in the above HPMC dispersion with continuous mixing. The predetermined solution containing ozanimod and HPMC were applied on to sugar spheres using a fluidized bed processor. After the completion of coating, the granules were dried under vacuum at 40° C. until desired loss on drying (LOD) was achieved. The dried granules were coated with seal coating solution comprising hydroxypropyl cellulose and the pellets were dried until desired LOD was achieved. Hydroxypropylmethyl cellulose phthalate (HPMC Phthalate), triethyl citrate and talc was dispersed in methanol-water solvent to obtain a dispersion (HPMC phthalate dispersion). The seal coated granules were coated with above prepared HMMC phthalate dispersion. The coated granules were dried until desired LOD was achieved. The granules were filled in hard gelatin capsules.

TABLE 10

| Ingredients | Amount | |
|---|---|---|
| Sugar Spheres NF | 125 | mg |
| Drug Layering | | |
| Ozanimod | 1.0 | mg |
| HPMC 5 cps | 5.0 | mg |
| Methanol | q.s. | |
| Purified Water | q.s. | |
| Weight of Drug Layered Pellets | 131.00 | mg |
| Seal Coating | | |
| Hydroxypropyl Cellulose | 7.0 | mg |
| Purified Water | q.s. | |
| Weight of Seal Coated Pellets | 138.00 | |
| Delayed-Release Layer | | |
| HPMC Phthalate | 37.5 | mg |
| Triethyl Citrate | 3.75 | mg |
| Talc | 7.5 | mg |
| Isopropyl Alcohol | q.s. | |
| Purified Water | q.s. | |
| Weight of DR Pellets | 186.78 | mg |

DR Pellets were filled in empty gelatin capsules

### Example 11: Immediate-Release Capsules Composition

Ozanimod hydrochloride was dissolved in methanol to make a clear solution. Microcrystalline cellulose and Croscarmellose sodium were fluidized together in a fluid bed processor. The drug solution was sprayed onto fluidized blend in a fluid bed processor. The obtained granules were dried and passed through a suitable screen to achieve uniformly sized granules. The granules were filled into capsules.

TABLE 11

| Ingredients | Amount | |
|---|---|---|
| Ozanimod Hydrochloride | 1.0 | mg |
| Microcrystalline cellulose | 193.0 | mg |
| Croscarmellose sodium (Ac-di-sol) | 6.0 | mg |
| Methanol | q.s. | |
| Total Weight | 200.00 | mg |

### Example 12: Pharmacokinetic/Pharmacodynamic and Bioavailability of Ozanimod from Immediate-Release and Delayed-Release Composition

The applicant has conducted a parallel group single dose PK study in rat to characterize the pharmacokinetic profile of the delayed-release (DR) formulation of Ozanimod (Exp. 10) in comparison with the immediate-release formulation of Ozanimod (in-house formulation, Exp. 11). In this study, Ozanimod as well as major active metabolite CC112273 were measured with LC-MS/MS method. Approximately 1 mg/kg and 0.89 mg/kg single dose was administered through oral gavage route for IR and DR formulations, respectively. The study data suggest that the dose normalized Cmax and AUClast of the delayed-release formulation for ozanimod analyte were approximately 52% and 14% lower than the immediate-release formulation. Similarly, the dose normalized Cmax and AUClast of delayed-release formulation and immediate-release formulation for Ozanimode active metabolite, CC112273 analyte were approximately 42% and 24%, respectively. The median tmax for Ozanimod and for its active metabolite CC112273 was 24 hours from delayed-release formulation and was 1 hour from immediate release formulation. The pharmacokinetic parameters of both the formulations for Ozanimod and active metabolite CC112273 are summarized in the Table 12 and 13.

TABLE 12
Experiment 01: Pharmacokinetic parameters of Ozanimod after
oral administration of Immediate-Release formulation and Delayed-
Release formulations of Ozanimod in Wistar rat male

| | IR formulation of | DR formulation of |
|---|---|---|
| | Ozanimod (Mean ± | Ozanimod (Mean ± |
| PK parameters | SD) | SD) |
| Formulation | Example 11 | Example 10 |
| Dose (mg/kg) | 1 | 0.89 |
| Tmax (h)* | 1.0 (1.0-2.0) | 24.0 (6.0-24.0) |
| Cmax (pg/mL) | 20913.14 ± | 8937.52 ± |
| | 8832.46 | 5205.66 |
| AUClast (pg*h/mL) | 205157.67 ± | 157340.21 ± |
| | 18042.19 | 53397.01 |
| DNCmax | 20913 | 10042 |
| DNAUC (pg*h/mL) | 205157 | 176786 |
| Cmax fold change (DR/IR) | — | 0.48 |
| AUClast fold change (DR/IR) | — | 0.86 |

*Median (range)

TABLE 13
Experiment 01: Pharmacokinetic parameters of active
metabolite CC112273 after oral administration of
Immediate-Release formulation and Delayed-Release
formulations of Ozanimod in Wistar rat male

| | IR formulation of | DR formulation of |
|---|---|---|
| | Ozanimod (Mean ± | Ozanimod (Mean ± |
| PK parameters | SD) | SD) |
| Formulation | Example 11 | Example 10 |
| Dose (mg/kg) | 1 | 0.89 |
| Tmax (h) | 1.0 (1.0-12.0) | 24.0 (6.0-24.0) |
| Cmax (pg/mL) | 1998.68 ± | 1040.07 ± |
| | 909.90 | 750.99 |
| AUClast (pg*h/mL) | 28658.77 ± | 19304.69 ± |
| | 8474.56 | 11056.71 |
| DNCmax | 1999 | 1169 |
| DNAUCt | 28659 | 21387 |
| Cmax fold change (DR/IR) | — | 0.58X |
| AUClast fold change (DR/IR) | — | 0.75 |

*Median (range)

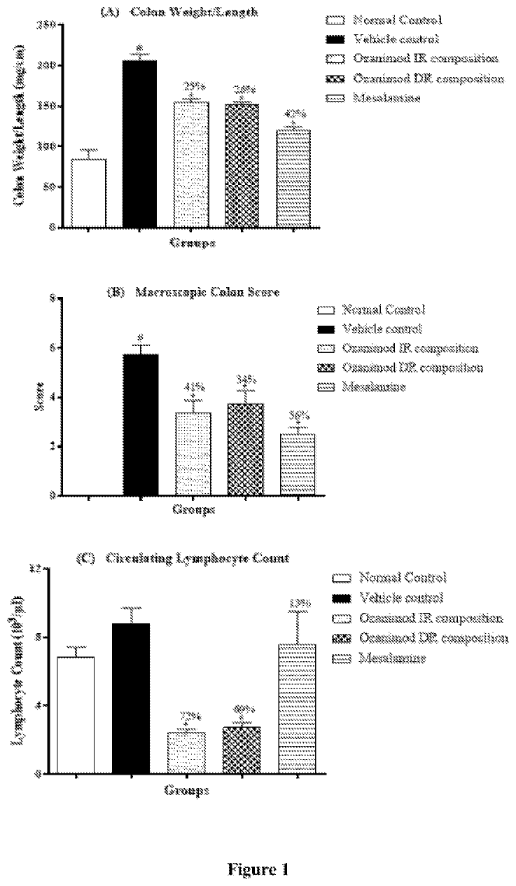

However, these differences in PK parameters were not observed in Pharmacodynamic (PD) study (Experiment 02) conducted in TNBS induced colitis model in rat. The PD study was performed at 1 mg/kg for IR formulation and 0.89 mg/kg for DR formulations as once in a day dose, administered through oral gavage route for 6 consecutive days. In this study, the efficacy parameters measured were colon weight to length ratio, colonic microscopic score (which includes sum of Adhesion score, Stricture score, Ulcer, Wall thickness) and Lymphocyte count. Colon weight to length ratio and macroscopic colon disease score were determined at the end of the study, whereas Lymphocyte count in circulation relative to vehicle treated TNBS challenged diseased animals (Vehicle control) was assessed 24 h after the last dose. Both the formulations (i.e., Delayed-Release formulation and Immediate-Release formulation) have demonstrated similar level of efficacy responses for all these parameters. The all three pharmacodynamic parameters of both the formulations are summarized in the Table 14 and FIG. 1. The reasons for the similar efficacy response even at the differences in PK profile between both the formulations could be due to receptor saturation of S1P1 receptors overexpressed at the intestine at the dose of 1 mg/kg administered in both the treatment groups. Reduced blood exposure suggests that more drug has been remains at localized site i.e., lower parts of gastrointestinal tract, after administration of the DR formulation resulted into similar efficacy to that of the IR formulation. Moreover, reduced blood levels indicates that delayed-release formulations also result into better safety profile specifically adverse effects associated with Cmax at higher dose. The cardiac side effects such as Bradyarrhythmia and Atrioventricular Conduction Delays, which are associated with high Cmax from immediate-release formulation, get reduced or eliminated with the use of delayed-release formulation without affecting efficacy. Thus, the delayed-release formulation may also offer the skipping or reduce frequency of up-titration scheme.

TABLE 14
Experiment 02: Pharmacodynamic parameters of Ozanimod after oral administration
of IR formulation and DR formulations of Ozanimod in Wistar rat male

| | | | Colon Macroscopic | | | |
|---|---|---|---|---|---|---|
| | | | Score [Sum of scores | | | |
| | Colon Weight to | | of Adhesion score, | | | |
| | Length ratio | | Stricture score, | | Lymphocyte count | |
| | (mg/cm) | | Ulcer, Wall thickness] | | (10³/μL) | |
| Treatment | Observation | % Inhibition | Observation | % Inhibition | Observation | % Inhibition |
| group/Efficacy | (Mean ± | (Mean ± | (Mean ± | (Mean ± | (Mean ± | (Mean ± |
| parameters | SEM) | SEM) | SEM) | SEM) | SEM) | SEM) |
| Vehicle | 206.77 ± 7.08 | NA | 5.75 ± 0.37 | NA | 8.77 ± 0.91 | NA |
| Control | | | | | | |
| IR | 154.75 ± 4.02 | 25.16 ± 1.94 | 3.38 ± 0.5 | 41.30 ± 8.66 | 2.43 ± 0.19 | 72.27 ± 2.14 |
| formulation | | | | | | |
| dose: | | | | | | |
| 1 mg/kg; | | | | | | |
| Oral) | | | | | | |
| DR | 152.18 ± 3.15 | 26.4 ± 1.52 | 3.75 ± 0.53 | 34.78 ± 9.15 | 2.71 ± 0.29 | 69.11 ± 3.31 |
| Formulation | | | | | | |
| Dose: | | | | | | |
| 0.89 mg/kg; | | | | | | |
| Oral) | | | | | | |
| Mesalamine | 120.44 ± 4.22 | 41.75 ± 2.04 | 2.5 ± 0.29 | 56.52 ± 5.02 | 7.6 ± 1.9 | 13.39 ± 21.66 |
| (150 mg/kg; | | | | | | |
| Oral) | | | | | | |
| Normal | 84.25 ± 11.67 | NA | NA | NA | 6.81 ± 0.61 | 22.34 ± 6.95 |
| control | | | | | | |

While the invention has been described in terms of its specific embodiments, certain modifications and equivalents will be apparent to those skilled in the art and are intended to be included within the scope of the invention.

1. A delayed-release pharmaceutical composition comprising ozanimod or a pharmaceutically acceptable salt thereof, one or more controlled release substances, and one or more pharmaceutically acceptable excipients, wherein the controlled release substance comprises one or more of pH dependent polymers and pH independent polymers.

2. The delayed-release pharmaceutical composition according to claim 1, wherein the composition releases ozanimod at a pH of about 5 to about 7.5.

3. (canceled)

4. The delayed-release pharmaceutical composition according to claim 1, wherein the pH dependent polymer is selected from one or more of cellulose derivatives such as hydroxypropyl methylcellulose phthalate, hydroxypropyl methylcellulose acetate succinate, cellulose acetate phthalate, and acrylate polymers such as methacrylic acid and methyl methacrylate-based polymers.

5. The delayed-release pharmaceutical composition according to claim 1, wherein the pH independent polymer is selected from one or more of polyvinyl alcohol, polyvinyl acetate, copolymer of polyvinyl acetate and polyvinylpyrrolidone, polymethacrylic acid derivatives, cellulose derivative, polyethylene oxide; chitosan and its derivative, gums, polysaccharides, lipids, waxes, cyclodextrins, and polyols.

6. The delayed-release pharmaceutical composition according to claim 1, wherein the one or more pharmaceutically acceptable excipients comprise one or more of diluents, binders, disintegrants, and lubricants.

7. The delayed-release pharmaceutical compositions according to claim 1, wherein the composition is in the form of a capsule, a tablet, granules, powder, pellets, minitablets, or a sachet.

8. The delayed-release pharmaceutical composition according to claim 1, wherein the controlled release substance is in a matrix with ozanimod or a pharmaceutically acceptable salt thereof.

9. (canceled)

10. (canceled)

11. (canceled)

12. (canceled)

13. (canceled)

14. A delayed-release pharmaceutical composition comprising a multi-particulate formulation comprising ozanimod or a pharmaceutically acceptable salt thereof, one or more controlled release substances, and one or more pharmaceutically acceptable excipients, wherein the composition release ozanimod at a pH of about 5 to about 7.5.

15. The pharmaceutical composition according to claim 14, wherein the controlled release substance comprises one or more of 1) pH dependent polymers selected from one or more of cellulose derivatives such as hydroxypropyl methylcellulose phthalate, hydroxypropyl methylcellulose acetate succinate, cellulose acetate phthalate, and acrylate polymers such as methacrylic acid and methyl methacrylate based polymers, and 2) pH independent polymers selected from one or more of polyvinyl alcohol, polyvinyl acetate, copolymer of polyvinyl acetate and polyvinylpyrrolidone, polymethacrylic acid derivatives, cellulose derivative, polyethylene oxide, chitosan and its derivative, gums, polysaccharides, lipids, waxes, cyclodextrins, and polyols.

16. (canceled)

17. A method of treating irritable bowel disease in a patient in need thereof, comprising administering to a patient an effective amount of a pharmaceutical composition according to claim 1.

18-31. (canceled)

32. The delayed-release pharmaceutical composition according to claim 1, wherein the controlled release substance is coated on a core comprising ozanimod or a pharmaceutically acceptable salt thereof.
